(19) United States
(12) Patent Application Publication
(10) Pub. No.: US 2012/0234329 A1
(43) Pub. Date: Sep. 20, 2012

(54) SURGICAL, THERAPEUTIC, OR DIAGNOSTIC TOOL

(75) Inventors: Wilfried Vancraen, Huldenberg (BE); Louis James Keppler, Strongsville, OH (US); Ben Geebelen, Haasrode (BE); Jeroen Dille, Kessel-Lo (BE)

(73) Assignee: Materialise N.V., Leuven (BE)

(21) Appl. No.: 13/395,247

(22) PCT Filed: Sep. 13, 2010
(86) PCT No.: PCT/EP2010/063399

§ 371 Date: Mar. 9, 2012

Related U.S. Application Data
(60) Provisional application No. 61/242,945, filed on Sep. 16, 2009.

(30) Foreign Application Priority Data
Sep. 11, 2009 (GB) 0915948.4

(51) Int. Cl. A61B 19/00

(52) U.S. Cl. 128/845

(57) ABSTRACT

The present invention relates to a therapeutic, diagnostic or surgical tool, e.g. surgical guide frame for medical treatments, comprising: —a rigid structure, and —at least one functional guidance element (13), wherein the rigid structure comprises means for positioning and holding the rigid structure around a soft tissue area of a patient and includes means for compressing the soft tissue area surrounded by the positioning and holding means as well as to the use of such a therapeutic, diagnostic or surgical tool for medical treatments, especially in areas where the therapeutic, diagnostic or surgical tool, e.g. surgical guide is supported on soft tissue.

### TECHNICAL FIELD OF THE INVENTION

The present invention relates to a surgical, therapeutic or diagnostic tool, e.g. a surgical compression guide for medical treatments, and methods of making and using the same.

It applies more particularly, but not exclusively, to a surgical, therapeutic, or diagnostic tool, e.g. a surgical guide, for computer aided and computer planned treatments in areas where the guide is supported on soft tissue as well as methods of making and using the same.

### BACKGROUND OF THE INVENTION

At present, there exists an increasing amount of surgical interventions that benefit from the use of medical image based patient specific surgical guides as described in patent applications US 2005/0203528 A1 and EP 1 486 900 A1, for instance.

These guides mostly support on bone or teeth or areas of the body that have a good rigidity. In some situations soft tissue guides are used that support on soft tissue, but always in areas of the body where the soft tissue is relatively thin and there is a good rigid bone base underneath. These guides are accurate when the soft tissue is thin on top of 3D-curved bony area such as is the case for soft tissue guides for oral implant placement that support on the pallatum. Typically, the current soft tissue guides are pressed in place by the surgeons or one of the surgical assistants. In many cases they are locked in place by putting a screw to the soft tissue, thus making a penetration through the skin and into the bone, creating a cavity which is not used for any further purpose during surgery.

A second approach to referencing surgical targets for which traditional guiding methods are difficult is to use a first reference surface which can use a traditional guiding method, as described in EP 1 486 900 A1, to position a device which can act over a surgical target. An example of this method is described in GB 2213066 A which uses the teeth or palate as the locking surface to reference surgical targets elsewhere in the mouth. This method allows for imaging of the surgical target such that anatomical features can be identified and the coordinates subsequently used for guiding surgical instruments. However, this technique is limited to areas in which the contact surface provides adequate fixation for unique positioning of the device and which have a fixed position relative to the underlying anatomy.

Unfortunately there are situations, for instance for the placement of intercalary femur implants, where the current generation of soft tissue guides can not be used. The muscle tissue in those regions is so thick that supporting on the soft tissue in the neighbourhood where a resection or a biopsy or another surgical act needs to be performed accurately is impossible. Even a quite large soft tissue guide that is placed on a considerable area of the tight muscles does not feel stable after placement. The situation can be further complicated when attempting to guide over a joint, e.g. knee or elbow, since it introduces additional degrees of freedom between the fitting surfaces. Unique positioning will often be a problem on these regions, especially with obese patients.

The result is that multiple surgical acts in those regions are difficult to perform in a guided way and therefore rely on the surgeon's mental and visual navigation. In some medical conditions, medical image based robot navigation or electronic navigation may help the surgeon. However, the electronic navigation systems rely on the accurate registration of the medical image datasets in which planning is done as well as accurate manual placement of the navigation's anatomic positioning markers. This can be problematic for the same reasons as those that affect the fit of mechanical guides, but also due to the fact that the position of the body of the patient may be quite different on the operation table as during the time that the scans were taken.

Triano et al. propose a solution for improving the accuracy of navigation over unstable fitting regions in US 2006/064005 A1. This is accomplished by fitting a brace around the soft tissue region, e.g. the hip and torso, and distracting the region such that it becomes, in effect, a rigid structure such that any movement of the body will maintain the target site in a fixed relationship to a specific position on the brace. Once this position is determined, the navigation system can use the position on the brace to further locate other navigated landmarks. While this approach manages to restrict the position of a soft tissue region to a known position, the initial brace does not fit onto a predefined patient specific position, and relies on the technologies inherent to electronic surgical navigation to relate its position to the patient's anatomy. Due to its lack of unique positioning, the brace also cannot be used during imaging to relate its position to the patient's anatomy for pre-operative planning. In this sense, it suffers from the same limitations as electronic navigation as described above.

An alternative approach to the problem is to perform some surgical acts that need high accuracy while the patient is being scanned. Theoretically this could give the best guiding feedback to the surgeon, because he can see in real time where he is positioned subcutaneously.

An example for such a method relating to a surgical act performed while scanning the patient can be found in U.S. Pat. No. 5,682,890. Here, a method of soft tissue stereotaxy is described that uses a flexible material which is softened, shaped and contoured to the soft tissue region which is then cured or hardened. With this method, a stiff exoskeleton is produced which can be fastened to the patient's corresponding supporting surface which can hold markers for the scanning procedure as well as guide the surgical instruments.

Unfortunately, this approach is expensive and has a large number of potential problems or inconveniences that need to be taken into account. For example, most 3D-image scanners leave insufficient room for the surgeon to operate. In addition, both the patient and the clinicians can be exposed to excessive radiation. In general when this approach is used it is mostly done with 2D X-ray imaging which gives only partial positioning information and still has the drawback of emitting radiation.

While the currently existing devices can help position the surgeon's instrumentation, most of these current methods do not allow for pre-operative planning which can be directly transferred to the surgical theatre. Some navigation systems allow pre-operative plans to be imported and followed intra-operatively, however, its accuracy is still limited by anatomic marker place and image registration as stated previously. The creation of form fitting guides allows for a direct transfer of the pre-operative plan to the patient but this technology is limited by the quality of the guide's fit. Over soft tissue, where a unique locking position is often not possible using solely a surface contour, a more accurate position can be obtained by the methods described in the invention.

### SUMMARY OF THE INVENTION

An object of the present invention is to provide a surgical compression guide for medical treatments, and methods of making and using the same. In particular it is an object of the present invention to provide a surgical guide for image based computer planned treatments in areas where the guide is supercutaneously supported on soft tissue and targets the subcutaneous anatomy.

Advantages of the present invention include provision of a guide that does not severely obstruct the surgeon, e.g. that gives accurate guidance while decreasing the invasiveness of the surgery or fixes the patient in a position that eases accurate surgery.

This object is accomplished by a surgical compression guide according to the present invention.

The present invention provides a mechanical reference for a surgical, therapeutic or diagnostic tool, e.g. a surgical guide frame for medical treatments, comprising:
a rigid structure, and
at least one functional guidance element,
first means for positioning and holding the rigid structure by compression, the first means for positioning and holding the rigid structure by compression being at a first location on the rigid structure and including a first surface, that is patient specific, having a negative form of a first part of a body of a patient, and further including a first compression means for holding the first surface against the first part of the body of the patient, and
second means for positioning and holding the rigid structure by compression about a second part of the body of the patient, the second means for positioning and holding the rigid structure by compression being at a second location on the rigid structure remote from the first location, the second means for positioning and holding including a second surface, that is patient specific, having a negative form of at least a portion of the second part of the body of the patient, and further including a second compression means for holding the second surface against the second part of the body of the patient.

The first surface can be the same as or an extension of the second surface. Either the first or second means for positioning and holding the rigid structure by compression may be for positional clamping the first or second surface to the first or second part of the body, respectively. Preferably at least one of the first or second means for positioning and holding the rigid structure by compression is for positional clamping of the first or second surface respectively to the first or second part of the body, respectively. It is to be noted that the terminology “first and second” does not relate to a time based sequence in the actual applications. The term “positional clamping” refers to a clamping device which is designed to clamp to a part of the body with subcutaneous bone onto which a stable clamping can be made, so that a stable position is obtained. The primary location device is whichever of the first and second means for positioning and holding the rigid structure which allows positional clamping. Preferably, the surface corresponding to the primary location device (e.g. the first surface) is patient specific, having the negative form of the concerned part of the body of the particular patient. Alternatively, the first or second means for positioning and holding the rigid structure may be designed to locate onto cutaneous soft tissue. The clamping that can be achieved is generally less precise than with positional clamping means because the underlying bone is covered by a thick layer of soft tissue such as skin, muscle and/or fat and hence is less stable than clamping to bone with a minimal covering of soft tissue. It is the combination of the first and second means that provides in combination with the rigid structure an overall accurate positioning of the guidance element by restricting the guide's degrees of freedom with respect to the body parts, not only at the skin level, but also the underlying bones and anatomic structures. By compressively registering onto bone, relative positions can be determined with respect to the patient's skeleton across any distance for which the skeleton maintains a known or fixed position. For some applications the first and second means will include multiple clamps residing on multiple surfaces. Further, if required a third, fourth, . . . means for positioning and holding may be used until all remaining degrees of freedom from the first and second means are eliminated and thus a unique and stable position is achieved.

It is the conscious use of clamping means designed to fit on surface areas of the skin where underlying rigidity can be accessed to limit certain degrees of freedom in the total guide device movement that discriminate the current invention from manually pressed or screwed soft tissue guides in the prior art. When the guide device extends over articulating body parts, i.e. joints, the means for positional clamping can be used to fix the articulating anatomy in a preoperatively defined position by their mechanism to eliminate degrees of freedom. In this instance, the distance and angle between the first and second means are set to uniquely position the guidance elements. The first and second means may be positioned on either side of the joint and the structure of the joint itself may act as either the first or second means of fixation.

The positioning of the rigid structure and the operation of the compression holding means used with the first and/or second means for positioning and holding the rigid structure, are non-invasive. For example, no screws or other invasive components are used, but rather enveloping compression clamps are employed. Hence, there is no need to place screws percutaneously into bone for placement purposes, nor to increase the incision size to expose an underlying bony surface onto which a traditional rigid surface guide, i.e. a perfected medical model as described in EP0756735B1, is mounted. The compression means used with the first and second means for positioning and holding the rigid structure, may apply compression by, for example, a circumferential clamp or a scissor clamp. For the circumferential clamp of the first or second means for positioning and holding the rigid structure, the compression can be achieved by different techniques, e.g. a strap may be used which wraps around the body. The compressive force can be measured and displayed such that a specific pressure can be planned or reproduced, e.g. by incorporating pressure pads into the patient-specific surface of the clamp, or by attaching a tension meter to the strap.

The first and/or the second surface may make direct contact with cutaneous soft tissue, under which lies bony anatomy.

In one embodiment of the present invention, the first and/or second surfaces can be generated by layered manufacturing, e.g. rapid prototyping manufacturing techniques, directly from medical images of the patient such as optical, MRI, PET-scan, CT-scan images, or Ultrasound images from which a surface can be generated, e.g. by segmentation. The first and/or second surfaces are patient specific corresponding to the negative form of a part of the patient's body. In this way the first and/or second surfaces register with the relevant part of the body, i.e. they are an exact surface match. In this embodiment, the patient wears the completed surgical, therapeutic, or diagnostic tool, e.g. a surgical guide frame, for the subsequent surgical, therapeutic, or diagnostic operation.

In another embodiment, a device without the functional guidance element is first attached to the patient and the patient is then imaged, e.g. with optical, MRI, PET-scan, CT-scan images, or Ultrasound imaging. Based on the images (which will show the device) the functional guide element is designed. The functional guidance element can be a navigation system, e.g. a trace that the surgeon is to follow, or the functional guidance element can be a physical guide such as a drill or surgical guide defining drill holes or cutting planes for example. The functional guidance element can be a reference marker for use with a navigational system. Also in this embodiment, the first and/or second surfaces can be generated by layered manufacturing, e.g. rapid prototyping manufacturing techniques, directly from medical images of the patient.

The rigid structure does not necessarily have a patient specific surface defined here as the negative form of a part of the patient's body. The rigid structure can be provided with fixing means at various locations to allow attachment of the functional guidance element (if present) and the first and second means for positioning and holding. The locations of the functional guidance element(s) and/or of the first and/or of the second means for positioning on the rigid structure may be defined preoperatively based on the surgical planning, using at least one medical image of the patient.

Accordingly, the present invention proposes a new design of surgical, therapeutic, or diagnostic tool, i.e. surgical guides, that can be based on using a medical image based mechanical guiding system that accurately fits over defined soft tissue areas of the patient's skin and allows precise targeting and control of surgical interventions. Due to the compression inherent in the design of the system, these guides do not need to be held in place by the surgeon or by percutaneous screws and pins which fixate the guide by penetrating into the bone thus creating a cavity which will not be used for any surgical purpose besides guide fixation.

The accurate fit is obtainable by positional clamping of the first and/or second means for positioning and holding the rigid structure that compresses onto bone through a minimal soft tissue layer, preferably non-invasively. Hence positioning or fixation elements of the first and/or second means for positioning and holding the rigid structure, fit on bony areas with a thin soft tissue coverage, e.g. the distal parts of the femur just above the knee. The rigid structure, which can be a frame, translates the accurate fit from the location of the positional clamp of one of the first or second means for positioning and holding to the position of the guidance element. The other of the first or second means for positioning and holding provides additional orientation assistance, i.e. stabilisation. It can provide a positional correction to the guidance element as determined by one of the first or second means for positioning.

In the current invention, a rigid structure such as a frame element is provided, and two or more positioning or fixation elements as means for positioning the frame element around a soft tissue area of a patient. The advantage of this rigid structure, e.g. frame, is that it provides mechanical references in areas with thick soft tissue as derived from a remote location where there is a bony clamping position. Additionally, the rigid structure or frame can also be used to bring the body or a part of it, e.g. the spine, into an exactly repeatable position, so that the relative positions of different body parts of the patients are identical during the image acquisition phase, the surgical planning phase in medical image data sets, and the surgical execution phase when the guided surgical act is performed.

Accordingly, the rigid structure, e.g. frame, assists in the relative positioning of functional guidance elements with respect to the first and second means of fixation over regions that are covered with thick layers of soft tissue. These guidance elements allow the surgeon to execute a therapeutic; diagnostic; or surgical act, e.g. insertion of a biopsy needle; placement of reference pins; drilling of holes; or surgical cutting, e.g. making osteotomy cuts accurately according to a predefined surgical plan; made using any kind of 3D-imaging technology, e.g. optical, CT, MRI, PET or Ultrasound. In some situations the functional guiding element may even comprise or interface with an electronic surgical navigation system or could be a reference for an electronic surgical navigation system.

The rigid structure, such as a frame, attached to the first and second means for positioning and holding the rigid structure around a soft tissue area of a patient, in conjunction with means for compressing the soft tissue area ensures the exact positioning of the guidance element. This may be achieved by the first means compressing some part of the body to provide a better fit to a body surface that is close to an underlying rigid body structure with the second means' positions restricting any remaining degrees of freedom. By compressing the underlying soft tissue to a known depth or force, the underlying rigid anatomy defines the softer tissue which is being compressed. This compression is preferably made by fully enveloping the region of interest with the first and second means for fastening and compressing.

Either one of the first and second positioning and holding means including fixation elements does not necessarily fix all degrees of freedom of the entire guiding system, but the combination of all positioning or fixation elements should ensure a unique and stable fit. For instance, the lateral side distal part of the femur can be used as a positioning or fixation area with two frame components, one component on each side of the femur. By enveloping the two components with a controlled wrap a stable and unique position of the guide compared to the femur can be obtained which removes the degrees of freedom that allow translation of the guide.

By combining multiple positioning or fixation elements on a rigid structure, e.g. a frame, overall stability and uniqueness can be achieved.

A rigid structure such as a frame element of the present invention is any device in which unique guide elements can be placed so that the unique parts are placed at a specific distance from each other or in reference to an anatomic structure. This can also be a mix of standard components with patient specific parts, i.e. parts with the negative form of a part of the patient's body. The frame elements can be adaptable to different body sections.

The frame element can be single use for one patient or multiple use. An example of a single use rigid structure or frame element is a plaster cast that is made before scanning the patient. It is preferred to make the rigid structure in components that are easy to remove. The alternative that the patient needs to wear the rigid structure from the day he is scanned until the surgery day is less preferred. Moreover, it makes the introduction of the guidance elements less elegant, though not unfeasible when they have to be fixed in the rigid structure while the patient is wearing it.

Normally, the components in plaster or another stiff moulding and modelling material that can be applied to the body will be constructed in two halves that are easy to remove and fit together again. The first and/or second positioning and holding means including positioning or fixation elements can have a positive tolerance to create the compression in the right areas where the exact positioning is taking place. Alternatives to casting such as expandable foam can also be used.

While it is possible to create large and comfortable rigid structures in this way for a patient, this approach has the disadvantage of being time consuming and expensive. Moreover, the casts have to be adapted to the consequent surgery so the rigid structure is modelled to allow the positioning of the guidance elements and the surgical approach. Consequently, while the use of mouldable material for the rigid structure is a feasible approach, it is not a preferred embodiment for most applications. In some applications, such as vertebra related applications for obese patients with extreme sizes, however, this may be the only economically feasible alternative.

By incorporating the methods for fixation of the surgical, therapeutic, or diagnostic tool, i.e. a surgical compression guide for medical treatments, as defined previously, a planned position of the guidance element can be achieved to provide surgical intervention. The guidance elements act by directing defined the surgical instrument's position, depth, and/or translational movement with respect to the fixed underlying anatomy. The guidance elements can be quite simple elements, such as holes that determine a drilling or injection direction and depth, or slots that allow a linear or non linear cutting operation. Or they can be more complex systems that allow movement in several restricted directions The guiding element can also be a complete navigation system that finds its stable reference located in the rigid structure described aboveln a preferred embodiment of the present invention the therapeutic, diagnostic, or surgical tool, i.e. a surgical compression guide according to the present invention, can be custom made for a single patient. Alternatively, it can be standard in a number of predefined sizes. The guidance elements can direct the surgical instrument percutaneously, e.g. an endoscope or biopsy needle; supercutaneously or to a position on the skin's surface, e.g. a scalpel; or to a subcutaneous target which has been surgically exposed. The guidance elements may either directly guide the surgical instrument or set a locked position a secondary means of positioning a traditional surgical intervention device, e.g. guiding the pins which position a standard cut block.

The rigid structure, e.g. in the form of frame elements, can be manufactured with an additive or layered manufacturing technique such as Rapid Prototyping or other additive fabrication technologies, or with classic CNC technologies.

Preferably, negative tolerances or cushioning components can be provided in components of the rigid structure.

These measures can be taken to avoid pinching or squeezing the skin of the patient, especially in those areas where different components of the entire therapeutic, diagnostic, or surgical tool, i.e. the surgical guide system, are mating.

In another preferred embodiment of the present invention the therapeutic diagnostic, or surgical tool, i.e. surgical compression guide according to the present invention, can be made from a material that is compatible with the scanning method used for planning and/or carrying out the therapeutic diagnostic or surgical intervention.

At least one of the means for positioning the rigid structure, e.g. frame element, around a soft tissue area of a patient are made of a limited number of radio-opaque fixed construction elements that are typical for a certain surgical intervention. For example, when CT is used as scanning method for the computer-aided planning of the surgery, the surgical guide can be made of Polyurethane foam elements of which some or all are mixed homogeneously with barium. The rigid structure can fit like an orthopaedic brace and can be worn comfortably by the patient during scanning. The Polyurethane foam elements can selectively be replaced by patient specific, i.e. having the negative form of a part of the patient's body, positioning or fixation elements and guidance elements that give the structure rigidity to place the relevant parts of the body in exactly the same position during the surgery as during the scanning. This would allow the medical image based planning to be safely reproduced within the surgical theatre.

Preferably, the means for compressing the soft tissue area can be an assembly of belts on the rigid structure that fit around the body.

Advantageously for the patient, it is possible to design the various elements of the therapeutic diagnostic or surgical tool, just on the basis of a normal medical image data set taken of the patient without any special device attached. In such a situation it is preferred to incorporate some positive tolerance on the mating surface of the rigid structure in order to compensate for the indentation of the soft tissue by the compression.

Alternatively, the means for positioning the rigid structure around a soft tissue area of a patient and means for compressing the soft tissue area can be produced by designing the components all around the body. It is then advised to use a negative offset in those areas where no compression is required. The parts of the rigid structure can be made of multiple rigid components that are screwed together or attached together with another mechanical method.

In another preferred embodiment of the present invention the means for compressing the soft tissue area comprise inflatable areas.

In this way, the pressure of compression can be controlled, for example by reading the pressure of the inflatable areas. Thus, it can be ensured that the pressure is identical during the scanning of the patient and his surgery when the patient is scanned with the inventive device already attached to his body.

According to another preferred embodiment of the invention the functional guidance element mates with specific details of the rigid structure.

The therapeutic diagnostic or surgical tool, e.g. surgical compression guide according to the present invention, is preferably used in medical treatments, especially in areas where the therapeutic diagnostic or surgical tool, e.g. surgical guide, is supported on soft tissue.

Examples of body zones that are eligible areas for using a therapeutic diagnostic, or surgical tool, e.g. surgical guide according to the present invention, are the skull, the orbits, the skull base, the top of the shoulders, the clavicle, the iliac crest of the pelvis, the distal part of the femur, the proximal part of the tibia, the frontal side of the tibia, the elbow joint, a multiplicity of areas in ankle and foot bones, and areas of the arms and hands for special treatments.

The present invention also includes a method of making the guides as described above, the method including an additive manufacturing technique such as rapid protoyping or layered manufacture.

The present invention also provides a method of attaching a surgical, therapeutic or diagnostic guide to a body of a patient, the guide having a rigid structure and for use with at least one functional guidance element, the method comprising the steps of:
positioning and compression holding at least the rigid structure at a first location on the rigid structure by a first surface that is a negative form of a first part of the body of the patient, and
positioning and holding the rigid structure by compression at a second location on the rigid structure remote from the first location about a second part of the body of the patient by a second surface that is a negative form of at least a portion of the second part of the body of the patient.

The positional and holding steps are preferably non-invasive. The reason for attaching can be for scanning, e.g. MRI, CT-Scan, Ultrasound scan, PET scan or similar.

Either of the first or second positioning and holding steps may include positional clamping to the first or second part of the body, respectively. Preferably at least one of the first or second positioning and holding steps is for positional clamping to the first or second part of the body, respectively. The term “positional clamping” refers to a clamping which is designed to clamp to a part of the body with subsurface bone onto which a stable clamping can be made. The primary location method step is whichever of the first and second positioning and holding steps allows positional clamping. Alternatively, the first or second positioning and holding steps may be designed to locate onto soft tissue. The clamping that can be achieved is generally less precise than with positional clamping because the underlying bone is covered by a thick layer of soft tissue such as skin, muscle and/or fat and hence is less stable than clamping to bone with a minimal covering of soft tissue. It is the combination of the first and second clamping steps that provides in combination with a rigid structure an overall accurate positioning of the guidance element with respect to the body parts, not only at the skin level, but also the underlying bones and organic structures. For some applications multiple clamping, residing on multiple surfaces can be used.

It is the conscious use of clamping designed to fit on surface areas of the skin where underlying rigidity can be accessed to limit certain degrees of freedom in the total guide device movement that discriminate the current invention from manually pressed or screwed soft tissue guides in the prior art. When the guide device extends over articulating body parts (joints), the positional clamping can be used to fix the articulations in a defined position by their mechanism to eliminate degrees of freedom.

The method may include making the guide by any additive manufacturing route such as layered manufacturing, e.g. a rapid prototyping process.

The method may include scanning the patient with the surgical, therapeutic or diagnostic guide in place and adding the functional guidance element after the scanning step.

Other features and advantages of the present invention will become apparent from the following detailed description, taken in conjunction with the accompanying drawings, which illustrate, by way of example, the principles of the invention.

### BRIEF DESCRIPTION OF THE DRAWINGS

FIGS. 1 to 4 show schematic views of a first embodiment of a surgical compression guide according to the present invention.

FIGS. 5 to 7 show schematic views of a further embodiment of a surgical compression guide according to the present invention.

FIGS. 8 to 10 show schematic views of yet a embodiment of a surgical compression guide according to the present invention.

FIG. 11 shows a schematic representation of a computer system that may be used with the present invention.

### DESCRIPTION OF THE ILLUSTRATIVE EMBODIMENTS

The present invention will be described with respect to particular embodiments and with reference to certain drawings but the invention is not limited thereto but only by the claims. The drawings described are only schematic and are non-limiting.

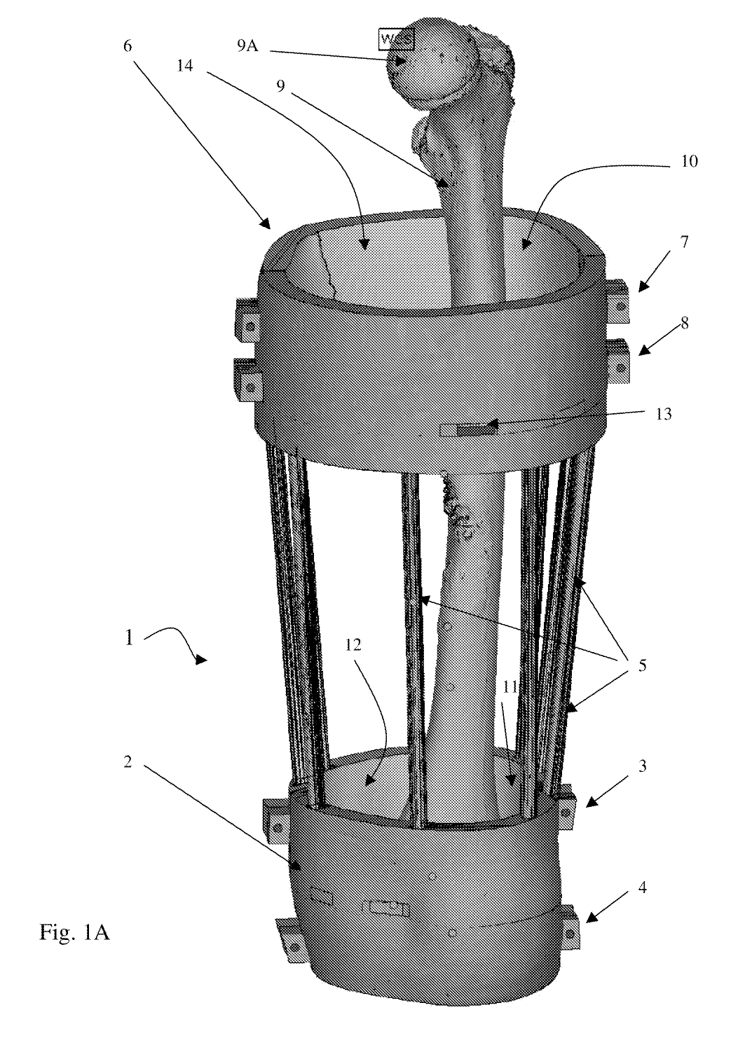

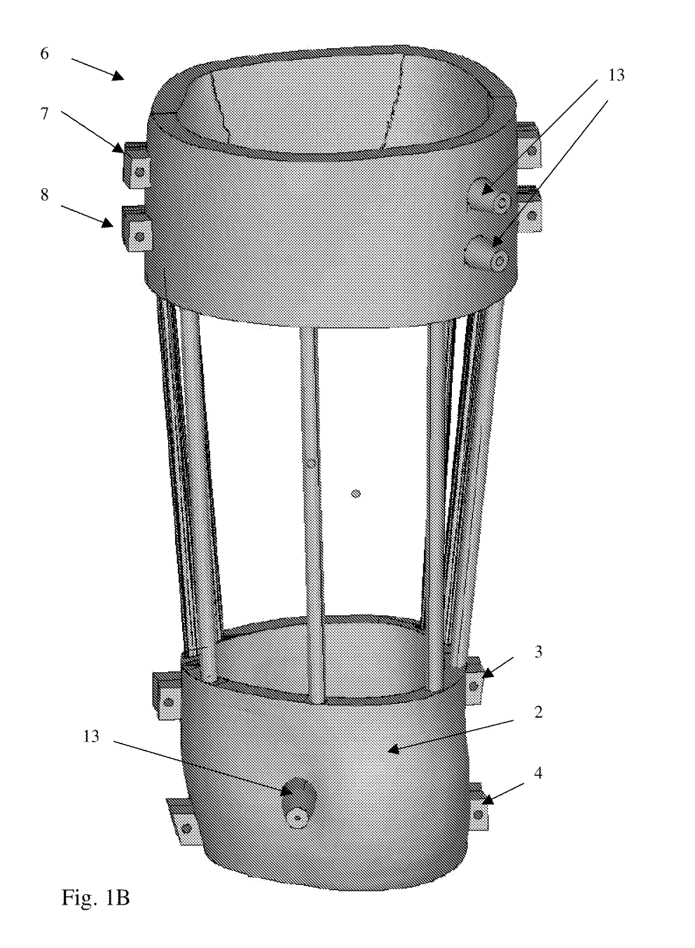

FIGS. 1A and 1B show schematic views of a surgical compression guide 1 according to the present invention.

A surgical compression guide 1 comprises a mechanical reference for a surgical, therapeutic or diagnostic tool, e.g. a surgical guide frame for medical treatments, in this case for surgery of the femur 9. A rigid structure 5 is provided, e.g. in the form of a frame. At least one functional guidance element 13 is provided. The guidance element 13 is fixed to the compression guide 1, e.g. optionally attachable directly or indirectly to the rigid structure 5 at a first location on the rigid structure. First and second means 6, 2 are provided for positioning and compression holding of the rigid structure 5 around parts of the body of the patient, e.g. around soft tissue areas of a patient. The guidance element 13 may be part of the first and second means 6, 2 that are provided for positioning and compression holding. The first and second means 6, 2 for positioning and compression holding the rigid structure have, respectively, a first (14) or second (12) surface that is a negative form of a part of the body of the patient, e.g. for around the knee and around the thigh. Compression means 6, 2 are provided for holding the first and/or second surface 14, 12, respectively against the relevant part of the body of the patient.

The first and second means 6, 2 for positioning and holding the rigid structure by compression are spaced apart from each and each is part of or attached to the frame 5. The first means 6 is at a first location on the rigid structure for fixing about a first part of the body of the patient e.g. thigh, whereas the second means for positioning and holding 2 is at a second location on the rigid structure remote from the first location and is for fixing about a second body part, e.g. knee. In a preferred embodiment, the first and second means for positioning and holding the rigid structure by compression are non-invasive. Also it is preferred if the surgical compression guide as a whole is non-invasive except for the operation that is to be carried out using the functional guidance element 13.

Either the first or second means 6, 2 for positioning and holding the rigid structure by compression may be for positional clamping the first or second surface respectively to the first or second part of the body, respectively. Preferably, at least one of the first or second means for positioning and holding the rigid structure by compression is for positional clamping of the first or second surface respectively to the first or second part of the body, respectively. The term “positional clamping” refers to a clamping device which is designed to clamp to a part of the body with subsurface bone onto which a stable clamping can be made. In this case the second means 6 is designed for fixing about the knee and as the skin or soft tissue is relatively thin over the knee, positional clamping can be achieved. Thus, the primary location device is, in this case, the second means 2 for positioning and holding the rigid structure which allows positional clamping. The first means 6 for positioning and holding the rigid structure is designed to locate onto the soft tissue of the thigh. The clamping that can be achieved is generally less precise than with positional clamping means because the underlying bone is covered by a thick layer of soft tissue such as skin, muscle and/or fat and hence is less stable than clamping to bone with a minimal covering of soft tissue. The first and second means preferably provide circumferential or enveloping clamping. It is the combination of the first and second means that provides in combination with the rigid structure an overall accurate positioning of the guide with respect to the body parts, not only at the skin level, but also the underlying bones and organic structures. The first and second means 6, 2 include first and second clamps 7,8; 3,4, respectively, that allow clamping of the surfaces 14, 12 respectively. The clamps 7,8; 3,4, are shown as joining two half surfaces that encircle or envelope the body part and are provided with flanges that can be used to force together the surfaces 14, 12 using screws, for example.

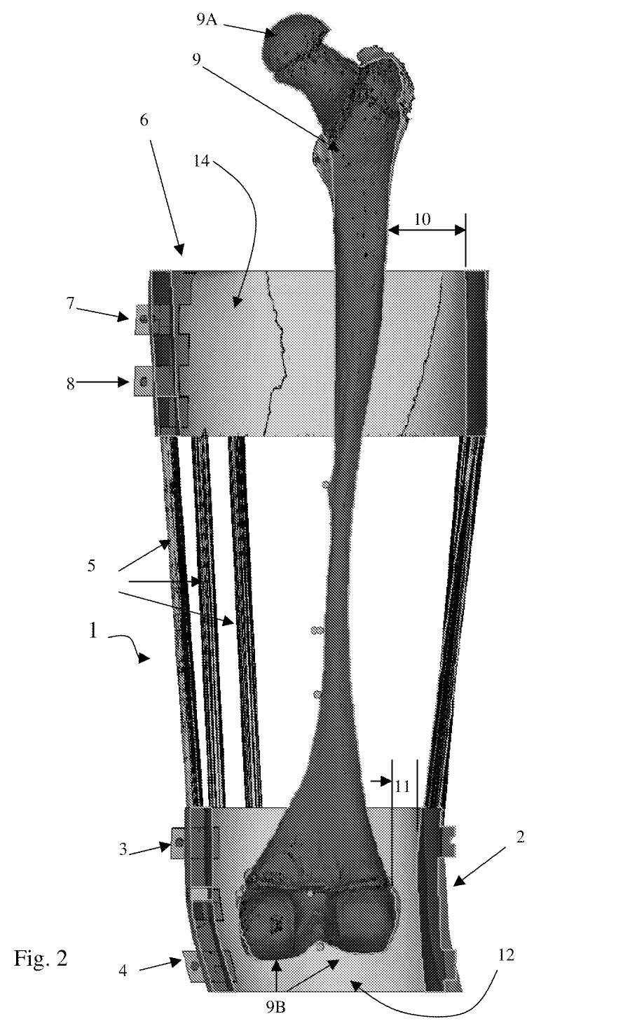

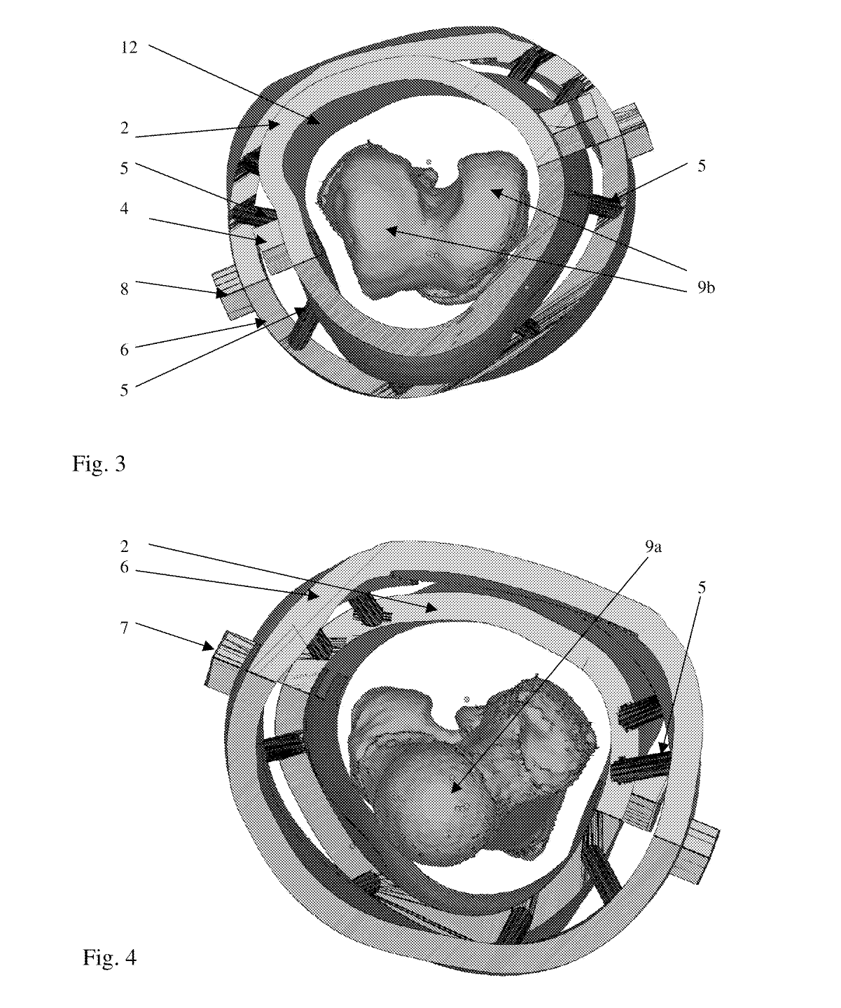

As shown in FIG. 2 (also in FIGS. 3 and 4) the distance 10 from the bone 9 to the first surface 14 of the first means for clamping 6 is much greater than the similar distance 11 between the bone 9B and the second surface 12 of the second means 2 for clamping. This is because there is a thick layer of muscle and fat in the thigh which is absent at the knee (soft tissue not shown).

The use of first and second clamping means 6, 2 designed to fit on surface areas of the skin where underlying rigidity can be used to limit certain degrees of freedom in the total guide device movement provides an advantage compared to manually pressed or screwed soft tissue guides in the prior art.

In one embodiment, the rigid structure or frame element 5 can be made with the clamping means 6 and 2 using moulding or plaster cast techniques. This can be made before scanning the patient in order to determine the position of the guide element 13. Plaster or another stiff moulding and modelling material is applied to the body to form two patient specific enveloping clamping regions (having the negative form of a part of the patient's body on the inside surface), e.g. around the thigh and the knee. The frame 5 in the form of rods, as well as the fixation means 3, 4, 7, 8 can be worked into the plaster or moulding material to form a complete rigid structure. The clamping regions can then be cut into two halves or into a larger number of pieces that are easy to remove and fit together again. The first and/or second positioning and holding means including the positioning or fixation elements 3, 4, 7, 8 can have a positive tolerance, e.g. by application of one or more layers of material to create the compression in the right areas where the exact positioning is to take place. Alternatives to casting such as expandable foam can also be used. In another preferred embodiment of the present invention the means for compressing the soft tissue area with a positive tolerance comprise inflatable areas. In this way, the pressure of compression can be controlled, for example by reading the pressure of the inflatable areas. Thus, it can be ensured that the pressure is identical during the scanning of the patient and his surgery when the patient is scanned with the inventive device already attached to his body.

The guide element 13 can be fabricated after scanning the patient with the rigid structure in place. Accordingly, the rigid structure, e.g. frame, acts as the basis for a well defined positioning of the functional guidance element despite the fact that the guidance can have to be carried out in or on body parts that are covered with thick layers of soft tissue. At least one of the means for positioning the rigid structure, e.g. frame element around a soft tissue area of a patient can be made of a limited number of radio-opaque fixed construction elements that are typical for a certain surgical intervention. For example, when CT is used as scanning method for the computer-aided planning of the surgery, the surgical guide 1 can be made of Polyurethane foam elements of which some or all are mixed homogeneously with barium. The rigid structure can fit like an orthopaedic brace and can be worn comfortably by the patient during scanning. The Polyurethane foam elements can selectively be replaced by patient specific (I.e. having the negative form of a part of the patient's body) positioning or fixation elements and guidance elements that give the structure rigidity to place the relevant parts of the body in exactly the same position during the surgery as during the scanning. This would allow the medical image based planning to be safely reproduced within the actual surgery.

The guidance elements can be quite simple elements with holes that determine a drilling or injection direction and optionally depth, or slots that allow a linear or non linear cutting or sawing operation. Or they can be more complex systems that allow movement in several restricted directions. The guidance element can also be a complete navigation system that finds its stable reference located in the rigid structure described above. In some situations the functional guidance element may interface with an electronic surgical navigation system or could be a reference for an electronic surgical navigation system.

The guidance element(s) allow to execute a therapeutic, diagnostic or surgical act such as insertion of a biopsy needle, placement of reference pins, drilling of holes, or surgical cutting such as making osteotomy cuts very accurately according to a predefined surgical plan made in any kind of 3D-imaging technology like optical, CT, MRI, PET or Ultrasound.

In another embodiment of the present invention, the first and/or second surfaces 14, 12 and first and second clamping means 6, 2 can be generated by additive or layered manufacturing, e.g. rapid prototyping manufacturing techniques directly from medical images of the patient such as optical, MRI, PET-scan, CT-scan images or Ultrasound images from which a surface can be generated, e.g. by segmentation. The first and/or second surfaces 14, 12 are hence patient specific, i.e. they have the negative form of a part of the patient's body. In this way the first and/or second surfaces 14, 12 register with the relevant part of the body, i.e. they are an exact match. In this embodiment, the patient wears the completed surgical, therapeutic or diagnostic tool, e.g. a surgical guide frame for the subsequent surgical, therapeutic or diagnostic operation.

The frame 5 and the guide element 13 can also be created by additive or layered manufacturing, e.g. rapid prototyping manufacturing techniques using medical images of the patient such as optical, MRI, PET-scan, CT-scan images or Ultrasound images as a guide. For example, the rigid structure, e.g. in the form of frame elements can be manufactured with an additive or layered manufacturing technique such as Rapid Prototyping or other additive fabrication technologies or with classic CNC technologies.

Optionally, negative tolerances or cushioning components can be provided in components of the rigid structure. These measures can be taken to avoid pinching or squeezing the skin of the patient, especially in those areas where different components of the entire therapeutic diagnostic or surgical tool, e.g. surgical guide system are mating.

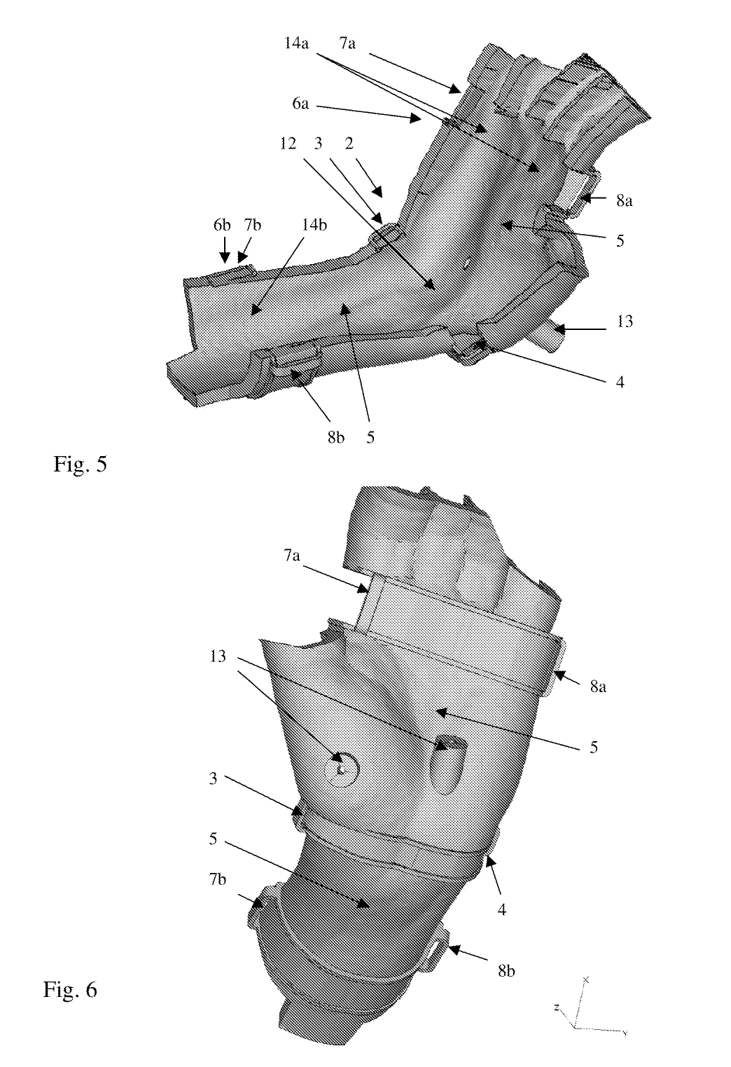

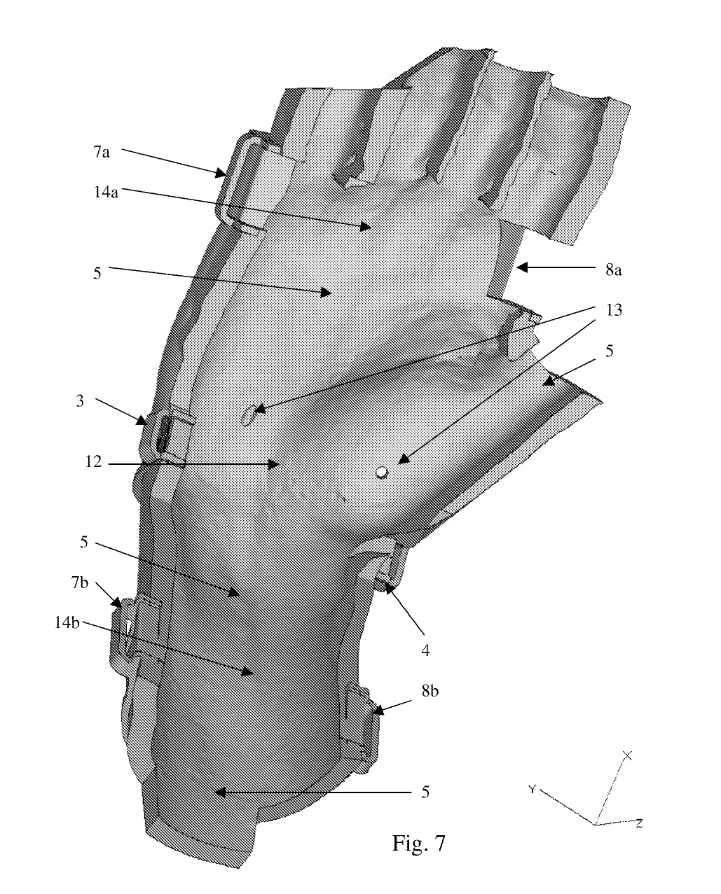

FIGS. 5 to 7 show a further embodiment of the present invention for use, for example to allow execution of a therapeutic, diagnostic or surgical act on the hand or wrist such as insertion of a biopsy needle, placement of reference pins, drilling of holes, or surgical cutting such as making osteotomy cuts very accurately according to a predefined surgical plan made in any kind of 3D-imaging technology like optical, CT, MRI, PET or Ultrasound.

In this embodiment, the rigid structure 5 is integral with first and second and third surfaces 14A, 12, 14B i.e. the rigid structure 5 is formed integrally with the surfaces 14A, 12, 14B. Also the hand of the patient is held at a specific position with respect to the wrist.

In accordance with this embodiment, a surgical compression guide 1 comprises a mechanical reference for a surgical, therapeutic or diagnostic tool, e.g. a surgical guide frame for medical treatments of the hand. A rigid structure 5 is provided, e.g. in the form of a patient specific surface, i.e. having the negative form of a part of the patient's body. At least one functional guidance element 13 is provided. The guidance element 13 is fixed to the compression guide 1, e.g. attached directly or indirectly to the rigid structure 5 at a first location on the rigid structure close to the wrist. The guide elements 13 are in this case drill guides. First, second and third means 6a, 2 and 6b, respectively are provided for positioning and compression holding of the rigid structure 5 around parts of the hand and wrist of the patient, e.g. around soft tissue areas of the patient. The first and second and third means 6a, 2, 6b for positioning and compression holding the rigid structure 5 have, respectively, a first (14a) or second (12) or third (14b) surface that is a negative form of the hand, fingers or wrist of the patient. Compression means 6a, 2, 6b are provided for holding the first and second and third surfaces 14a, 12, 14b, respectively against the relevant part of the body of the patient. The first, second and third compression means 6A, 2, 6B preferably provide circumferential or enveloping clamping.

The first and second and third means 6a, 2, 6b for positioning and holding the rigid structure by compression are spaced apart from each and each is integral with the frame 5. The first means 6a is at a first location on the rigid structure for fixing about a first part of the body of the patient e.g. the upper hand and fingers, whereas the second means for positioning and holding 2 is at a second location on the rigid structure remote from the first location and is for fixing about a second body part, e.g. at the junction of the wrist and the hand. In a preferred embodiment, the first, second and third means for positioning and holding the rigid structure by compression are non-invasive. Also it is preferred if the surgical compression guide is non-invasive except for the operation that is to be carried out using the functional guidance element.

The second means 2 for positioning and holding the rigid structure by compression is for positional clamping the second surface 12 to the second part of the body. In this case the second means 2 is designed for fixing about the junction of the hand and wrist as the skin or soft tissue is relatively thin over this region so that positional clamping can be achieved. Thus, the primary location device is, in this case, the second means 2 for positioning and holding the rigid structure which allows positional clamping. The first means 6A for positioning and holding the rigid structure is designed to locate onto the upper hand and fingers. The clamping that can be achieved is generally less precise than with positional clamping means because the fingers are flexible and less stable than clamping to bone with a minimal covering of soft tissue. The third means 6B for positioning and holding the rigid structure is designed to locate onto the wrist or lower arm. It is the combination of the first, second and third means that provides in combination with the rigid structure an overall accurate positioning of the guide with respect to the body parts, not only at the skin level, but also the underlying bones and organic structures. The first, second and third means 6A, 2, 6B include clamps 7A,8A; 3,4, 7B, 8B respectively, that allow clamping of the surfaces 14A, 12, 14B respectively. Clamps 7A,8A; 3,4; 7B, 8B are for use with straps that encircle or envelope the body part and can be used to force the surfaces 14A, 12, 14b against the matching body parts.

The use of first and second and third clamping means 6A, 2, 6B designed to fit on surface areas of the skin where underlying rigidity can be used to limit certain degrees of freedom in the total guide device movement provides an advantage compared to manually pressed or screwed soft tissue guides in the prior art.

In one embodiment, the rigid structure elements 5 can be made with the clamping means 6A, 2, 6B using moulding or plaster cast techniques. This can be made before scanning the patient in order to determine the position of the guide elements 13. Plaster or another stiff moulding and modelling material is applied to the hand when in a prescribed position with respect to the wrist to form a single patient specific enveloping surface (I.e. having the negative form of a part of the patient's body) onto which clamping regions are formed. The fixation means 3, 4, 7A, 8A, 7B, 8B can be worked into the plaster or moulding material to form a complete rigid structure. Alternatives to casting such as expandable foam can also be used.

The guide element 13 can be fabricated after scanning the patient with the rigid structure in place. Accordingly, the rigid structure, e.g. frame, acts as the basis for a well defined positioning of functional guidance element(s) 13 despite the fact that the guidance can have to be carried out in or on body parts that are covered with thick layers of soft tissue. At least one of the means for positioning the rigid structure, e.g. frame element around a soft tissue area of a patient can be made of a limited number of radio-opaque fixed construction elements that are typical for a certain surgical intervention. For example, when CT is used as scanning method for the computer-aided planning of the surgery, the surgical guide 1 can be made of Polyurethane foam elements of which some or all are mixed homogeneously with barium. The rigid structure can fit like an orthopaedic brace and can be worn comfortably by the patient during scanning. The Polyurethane foam elements can selectively be replaced by patient specific (i.e. having the negative form of a part of the patient's body) positioning or fixation elements and guidance elements that give the structure rigidity to place the relevant parts of the body in exactly the same position during the surgery as during the scanning. This would allow the medical image based planning to be safely reproduced within the actual surgery.

The guidance elements 13 can be quite simple elements with holes that determine a drilling or injection direction and optionally depth, or slots that allow a linear or non linear cutting or sawing operation. Or they can be more complex systems that allow movement in several restricted directions. The guidance element can also be a complete navigation system that finds its stable reference located in the rigid structure described above. In some situations the functional guidance element may interface with an electronic surgical navigation system or could be a reference for an electronic surgical navigation system.

In another embodiment of the present invention, the first and/or second and/or third surfaces 14A, 12, 14B and first, second and third clamping means 6A, 2, 6B can be generated by additive or layered manufacturing, e.g. rapid prototyping manufacturing techniques directly from medical images of the patient such as optical, MRI, PET-scan, CT-scan images or Ultrasound images from which a surface can be generated, e.g. by segmentation. The first and/or second and/or third surfaces 14A, 12, 14B are hence patient specific (i.e. having the negative form of a part of the patient's body). In this way the first and/or second and/or third surfaces 14A, 12, 14B register with the relevant part of the body, i.e. they are an exact match. In this embodiment, the patient wears the completed surgical, therapeutic or diagnostic tool, e.g. a surgical guide frame for the subsequent surgical, therapeutic or diagnostic operation.

The frame 5 and the guide element(s) 13 can also be created by additive or layered manufacturing, e.g. rapid prototyping manufacturing techniques using medical images of the patient such as optical, MRI, PET-scan, CT-scan images or Ultrasound images as a guide. For example, the rigid structure, e.g. in the form of frame elements can be manufactured with a additive or layered manufacturing technique such as Rapid Prototyping or other additive fabrication technologies or with classic CNC technologies.

Optionally, negative tolerances or cushioning components can be provided in components of the rigid structure. These measures can be taken to avoid pinching or squeezing the skin of the patient, especially in those areas where different components of the entire therapeutic diagnostic or surgical tool, e.g. surgical guide system are mating.

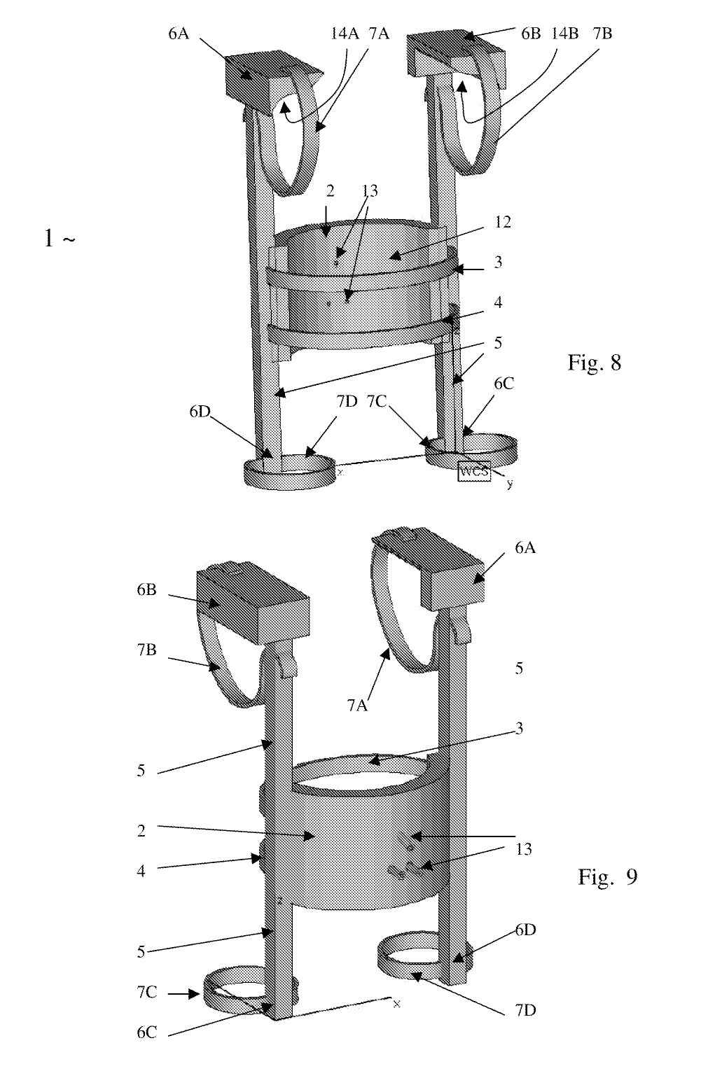

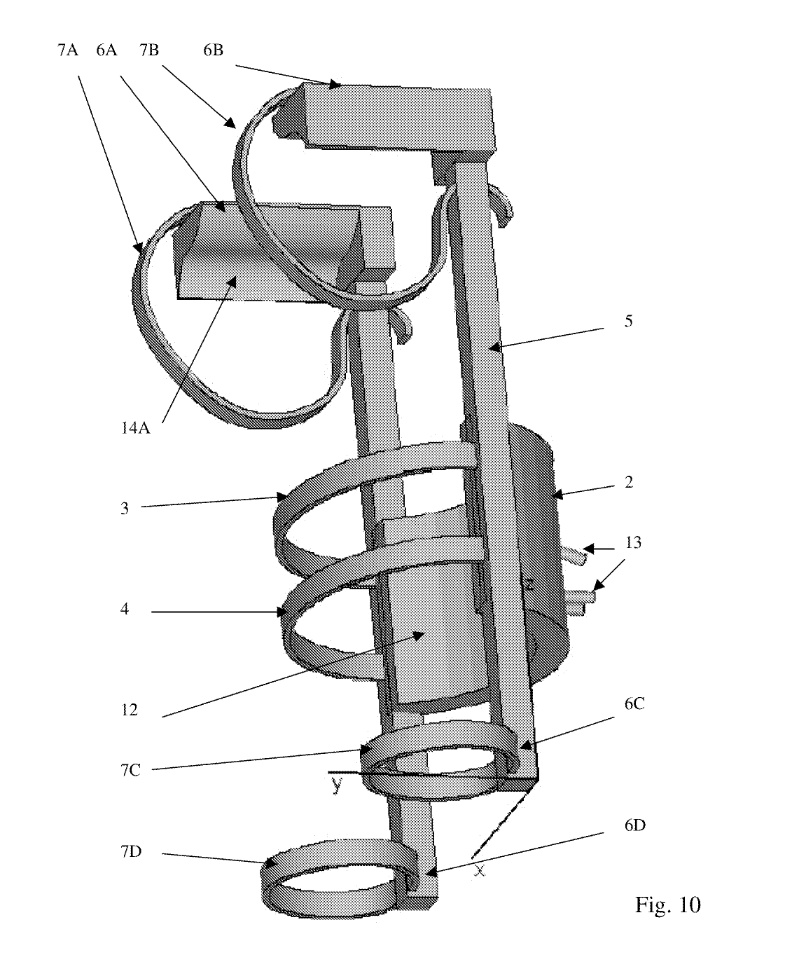

FIGS. 8 to 10 show a further embodiment of the present invention for use, for example to allow execution of a therapeutic, diagnostic or surgical act on the back or spinal region of a patient such as insertion of a biopsy needle, placement of reference pins, drilling of holes, or surgical cutting such as making osteotomy cuts very accurately according to a predefined surgical plan made in any kind of 3D-imaging technology like optical, CT, MRI, PET or Ultrasound.

In this embodiment, the rigid structure 5 is in the form of a frame that links mechanically with first and second and third surfaces 14A, 12, 14B and with clamps 6A, 6B, 2, 6C, 6D. Also the spine of the patient is held at a specific position.

In accordance with this embodiment, a surgical compression guide 1 comprises a mechanical reference for a surgical, therapeutic or diagnostic tool, e.g. a surgical guide frame for medical treatments of the back or spinal region. A rigid structure 5 is provided as well as at least one functional guidance element 13. The guidance element 13 is fixed to the compression guide 1, e.g. attached indirectly to the rigid structure 5 at a first location on the rigid structure close to the spine. The guide elements 13 are in this case drill guides. First, second, third, fourth and fifth means 6a, 2 and 6b, 6c, 6d respectively are provided for positioning and compression holding of the rigid structure 5 around parts of the back, shoulders and thighs of the patient, e.g. around soft tissue areas of the patient. The first and second, third means 6a, 2 and 6b respectively for positioning and compression holding the rigid structure 5 have, respectively, a first (14a) or second (12) or third (14b) surface that is a negative form of the back, or shoulders of the patient, respectively. Compression means 6a, 2, 6b are provided for holding the first and second and third surfaces 14a, 12, 14b, respectively against the relevant part of the body of the patient. In addition further compression means 6c and 6d are provided for fixing the frame 5 to the thighs of the patient. Thighs usually have a large thickness of fat and/or muscle and are hence less accurate for clamping purposes.

The first and second, third, fourth and fifth means 6a, 2, 6b, 6c, 6d for positioning and holding the rigid structure by compression are spaced apart from each and each is attached to the frame 5. The first means 6a is at a first location on the rigid structure for fixing about a first part of the body of the patient e.g. one shoulder, whereas the second means for positioning and holding 2 is at a second location on the rigid structure remote from the first location and is for fixing about a second body part, e.g. at the back of the patient. The third means 6b is at a third location on the rigid structure for fixing about a third part of the body of the patient e.g. the other shoulder, In a preferred embodiment, the first to fifth means for positioning and holding the rigid structure by compression are non-invasive. Also it is preferred if the surgical compression guide is non-invasive except for the operation that is to be carried out using the functional guidance element.

The first and third means 6A, 6B for positioning and holding the rigid structure by compression provide in combination positional clamping. Thus, the primary location device is, in this case, a combination of the first and third means for positioning and holding the rigid structure which allows positional clamping. The fourth and fifth means 6C, 6D for positioning and holding the rigid structure are designed to locate onto the upper thigh. The clamping that can be achieved with 6C and 6D is generally less precise than with positional clamping means because the thigh is typically covered with a thick layer of muscle and/or fat. It is the combination of the first to fifth means that provides in combination with the rigid structure an overall accurate positioning of the guide with respect to the body parts, not only at the skin level, but also the underlying bones and organic structures. The first to fifth means 6A, 2, 6B, 6C, 6D include clamps 7A; 3, 4; 7B; 7C; 7D respectively, that allow clamping of the surfaces 14A, 12, 14B to the shoulders and back and the frame 5 to the thighs. Clamps 7A; 3,4; 7B; 7C; 7D are use straps that encircle or envelope the body part and can be used to force the rigid structure against the matching body parts.

The use of first to fifth clamping means 6A, 7A; 2, 3, 4; 6B, 7B; 6C, 7C; 6D, 7D designed to fit on surface areas of the skin where underlying rigidity can be used to limit certain degrees of freedom in the total guide device movement provides an advantage compared to manually pressed or screwed soft tissue guides in the prior art.

In one embodiment, the rigid structure elements 5 can be made with the clamping means 6A, 2, 6B, 6C, 6D using moulding or plaster cast techniques. This can be fabricated and applied before scanning the patient in order to determine the position of the guide elements 13. Plaster or another stiff moulding and modelling material is applied to the back when in a prescribed position to form a patient specific enveloping surface (I.e. having the negative form of a part of the patient's body) onto which a clamping region is formed. The fixation means 3, 4, 7A, 7B, 7C, 7D can be attached to the frame 5 to form a complete rigid structure. Alternatives to casting such as expandable foam can also be used.

The guide element 13 can be fabricated after scanning the patient with the rigid structure in place. Accordingly, the rigid structure, e.g. frame, acts as the basis for a well defined positioning of functional guidance element(s) 13 despite the fact that the guidance can have to be carried out in or on body parts that are covered with thick layers of soft tissue. At least one of the means for positioning the rigid structure, e.g. frame element around a soft tissue area of a patient can be made of a limited number of radio-opaque fixed construction elements that are typical for a certain surgical intervention. For example, when CT is used as scanning method for the computer-aided planning of the surgery, the surgical guide 1 can be made of Polyurethane foam elements of which some or all are mixed homogeneously with barium. The rigid structure can fit like an orthopaedic brace and can be worn comfortably by the patient during scanning. The Polyurethane foam elements can selectively be replaced by patient specific (i.e. having the negative form of a part of the patient's body) positioning or fixation elements and guidance elements that give the structure rigidity to place the relevant parts of the body in exactly the same position during the surgery as during the scanning. This would allow the medical image based planning to be safely reproduced within the actual surgery.

The guidance elements 13 can be quite simple elements with holes that determine a drilling or injection direction and optionally depth, or slots that allow a linear or non linear cutting or sawing operation. Or they can be more complex systems that allow movement in several restricted directions. The guidance element can also be a complete navigation system that finds its stable reference located in the rigid structure described above. In some situations the functional guidance element may interface with an electronic surgical navigation system or could be a reference for an electronic surgical navigation system.

In another embodiment of the present invention, the first and/or second and/or third surfaces 14A, 12, 14B can be generated by additive or layered manufacturing, e.g. rapid prototyping manufacturing techniques directly from medical images of the patient such as optical, MRI, PET-scan, CT-scan images or Ultrasound images from which a surface can be generated, e.g. by segmentation. The first and/or second and/or third surfaces 14A, 12, 14B are hence patient specific (I.e. having the negative form of a part of the patient's body). In this way the first and/or second and/or third surfaces 14A, 12, 14B register with the relevant part of the body, i.e. they are an exact match. In this embodiment, the patient wears the completed surgical, therapeutic or diagnostic tool, e.g. a surgical guide frame for the subsequent surgical, therapeutic or diagnostic operation.

The frame 5 and the guide element(s) 13 can also be created by additive or layered manufacturing, e.g. rapid prototyping manufacturing techniques using medical images of the patient such as optical, MRI, PET-scan, CT-scan images or Ultrasound images as a guide. For example, the rigid structure, e.g. in the form of frame elements can be manufactured with an additive or layered manufacturing technique such as Rapid Prototyping or other additive fabrication technologies or with classic CNC technologies.

Optionally, negative tolerances or cushioning components can be provided in components of the rigid structure. These measures can be taken to avoid pinching or squeezing the skin of the patient, especially in those areas where different components of the entire therapeutic diagnostic or surgical tool, e.g. surgical guide system are mating.

The present invention includes for any of the embodiments that image based techniques may be used to create all or part of the guides according to embodiments of the present invention. A scanner such as an optical, CT-San, MRI, PET, X-ray, Ultrasound imaging device may be used to generate a digital 3D geometry of the relevant shape, e.g. one or more of the surfaces 12, 14 as obtained by scanning the relevant body part. The image can be in the form of a point cloud, a solid surface consisting of triangles or any other format for recording and storing a 3D geometry. Another way of obtaining the required geometry is to manually make a plaster cast of the body part such as limb and to capture the shape of the cast by any suitable technique, e.g. scanning. Alternatively, a positive made from the cast can be scanned.

The geometry of the body part determined can be digitally imported into a computer program and may be converted using algorithms known from the field of CAD/CAM technology to produce a 3D computer model of a relevant surface. A computer program such as 3-matic™ as supplied by Materialise N.V., Leuven, Belgium, may be used for constructing this 3D model. This geometry data can be used immediately in the computer program or stored in a digital file.

Once the 3D model of a surface, e.g. 12 or 14, is constructed, it may be manipulated manually, semi-automatically or automatically to design a 3D model of the relevant guide. These manipulations may include one or more of the following processes but are not limited to:
1. Scaling the geometry smaller or larger along certain axis.
2. Giving the geometry a thickness that can be varied throughout the part.
3. In creating hollow volumes inside this thickness.
4. Adding new surface shapes in certain parts, such as local elevations.
5. Adding predetermined 3D elements from a database system (E).
6. Integrating the interventions made into an optimal shape.
7. Adding attachment features that enable the attachment of straps or other means to fasten device to the person for whom it is designed.
8. Adding holes or other features.
   A preferred method for performing these actions uses a computer program such a 3-matic as supplied by Materialise N.V., Leuven, Belgium.

A data base library of one or more 3D models of relevant structures or their mathematical representations may then be used to incorporate at least one functional structure into the 3D model of the device, e.g. the rigid structure 5 or a guidance element 13 may be introduced. The elements in the library may be selected manually or automatically from the database by their pre-determined properties, such as their physical dimensions, their appearance or their mechanical properties. It is to be understood that the dimensions and values regarding the performance of all such imported structures available in the library may be scaled in any dimension to obtain the preferred or expected mechanical properties and performance. Functions representing them and their performance are preferably stored in this database so that they can be called up when required, automatically or manually by the user, and integrated into the 3D design using the design software. Specific structures may be called from the library or all structures matching certain performance parameters for the user to select for a particular location and purpose may be called. More than one structure can be selected by the library system to give certain areas of the device specific properties.

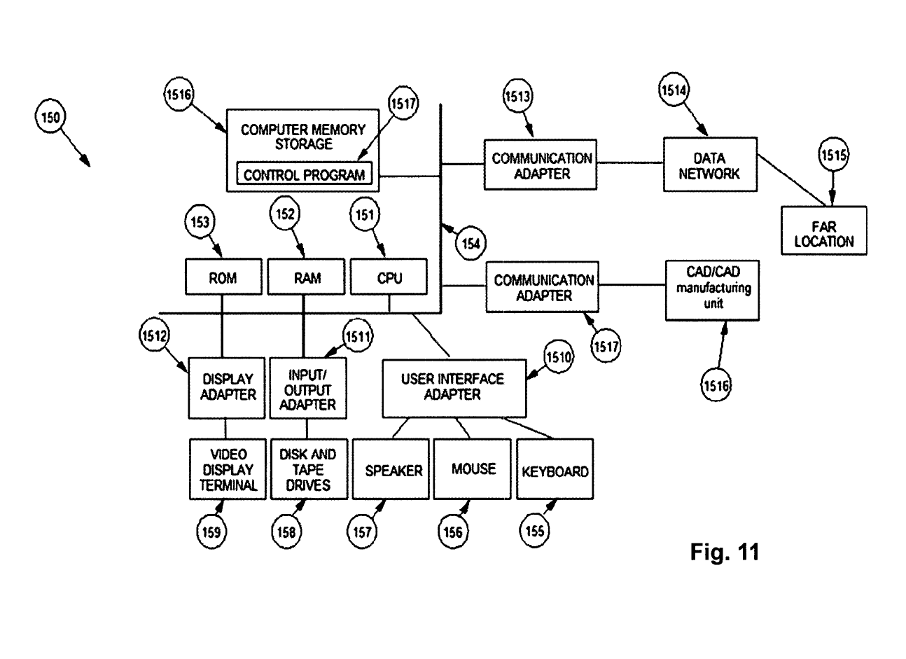

FIG. 11 is a schematic representation of a computing system which can be utilized with the methods and in a system according to the present invention including computer programs such as 3-matic™ as supplied by Materialise N.V., Leuven, Belgium. A computer 150 is depicted which may include a video display terminal 159, a data input means such as a keyboard 155, and a graphic user interface indicating means such as a mouse 156. Computer 150 may be implemented as a general purpose computer, e.g. a UNIX workstation or a personal computer.

Computer 150 includes a Central Processing Unit (“CPU”) 151, such as a conventional microprocessor of which a Pentium processor supplied by Intel Corp. USA is only an example, and a number of other units interconnected via bus system 154. The bus system 154 may be any suitable bus system—FIG. 11 is only schematic. The computer 150 includes at least one memory. Memory may include any of a variety of data storage devices known to the skilled person such as random-access memory (“RAM”), read-only memory (“ROM”), non-volatile read/write memory such as a hard disc as known to the skilled person. For example, computer 150 may further include random-access memory (“RAM”) 152, read-only memory (“ROM”) 153, as well as a display adapter 1512 for connecting system bus 154 to a video display terminal 159, and an optional input/output (I/O) adapter 1511 for connecting peripheral devices (e.g., disk and tape drives 158) to system bus 154. Video display terminal 159 can be the visual output of computer 150, which can be any suitable display device such as a CRT-based video display well-known in the art of computer hardware. However, with a desk-top computer, a portable or a notebook-based computer, video display terminal 159 can be replaced with a LCD-based or a gas plasma-based flat-panel display. Computer 150 further includes user interface adapter 1510 for connecting a keyboard 155, mouse 156, optional speaker 157. The relevant data describing the 3-D object to be formed may be input directly into the computer using the keyboard 155 or from storage devices such as 158, after which a processor carries out a method in accordance with the present invention. The results of the method may be transmitted to a further near or remote location, e.g. a CAD/CAM processing facility to manufacture the template in accordance with the details provided by computer 150.

A CAD/CAM manufacturing unit 1516 may also be connected via a communications adapter 1517 to bus 154 connecting computer 150 to a data network such as the Internet, an Intranet a Local or Wide Area network (LAN or WAN) or a CAN. The manufacturing unit 1516 may receive an output value or support descriptor file directly from computer 150 running a computer program for support design in accordance with the present invention or a value or descriptor file derived from such an output of computer 150. Alternatively, the unit 1516 may receive the relevant design data indirectly on a suitable signal storage medium such as a diskette, a replaceable hard disc, an optical storage device such as a CD-ROM or DVD-ROM, a magnetic tape or similar.

Computer 150 also includes a graphical user interface that resides within machine-readable media to direct the operation of computer 150. Any suitable machine-readable media may retain the graphical user interface, such as a random access memory (RAM) 152, a read-only memory (ROM) 153, a magnetic diskette, magnetic tape, or optical disk (the last three being located in disk and tape drives 158). Any suitable operating system and associated graphical user interface (e.g., Microsoft Windows, Linux) may direct CPU 151. In addition, computer 150 includes a control program 1517 that resides within computer memory storage 1516. Control program 1517 contains instructions that when executed on CPU 151 allow the computer 150 to carry out the operations described with respect to any of the methods of the present invention.

Those skilled in the art will appreciate that the hardware represented in FIG. 11 may vary for specific applications. For example, other peripheral devices such as optical disk media, audio adapters, or chip programming devices, such as PAL or EPROM programming devices well-known in the art of computer hardware, and the like may be utilized in addition to or in place of the hardware already described.

In the example depicted in FIG. 11, the computer program product for carrying out a method of the present invention can reside in any suitable memory. However, it is important that while the present invention has been, and will continue to be, that those skilled in the art will appreciate that the mechanisms of the present invention are capable of being distributed as a computer program product in a variety of forms, and that the present invention applies equally regardless of the particular type of signal bearing media used to actually carry out the distribution. Examples of computer readable signal bearing media include: recordable type media such as floppy disks and CD ROMs and transmission type media such as digital and analogue communication links.

Accordingly, the present invention also includes a software product which when executed on a suitable computing device carries out any of the methods of the present invention. Suitable software can be obtained by programming in a suitable high level language such as C and compiling on a suitable compiler for the target computer processor.

Having designed a surface and a support for the surface, the surface being a negative form of a body part, e.g. a surface 12 or 14, it can be manufactured, e.g. in one preferred embodiment, by additive manufacturing techniques. Additive Manufacturing (AM) can be defined as a group of techniques used to quickly fabricate a scale model of an object typically using three-dimensional (3-D) computer aided design (CAD) data of the object. The CAD/CAM manufacturing unit 1516 of FIG. 11 can be adpted for additive manufacturing techniques in order to construct any of the embodiments of the present invention. Currently, a multitude of Additive Manufacturing techniques is available, including stereo lithography (SLA), Selective Laser Sintering (SLS), Fused Deposition Modelling (FDM), foil-based techniques, etc.

Stereo lithography, presently the most common AM technique, utilizes a vat of liquid photopolymer “resin” to build an object a layer at a time. On each layer, an electromagnetic ray, e.g. one or several laser beams which are computer-controlled, traces a specific pattern on the surface of the liquid resin that is defined by the two-dimensional cross-sections of the object to be formed. Exposure to the electromagnetic ray cures, or, solidifies the pattern traced on the resin and adheres it to the layer below. After a coat had been polymerized, the platform descends by a single layer thickness and a subsequent layer pattern is traced, adhering to the previous layer. A complete 3-D object is formed by this process.

Selective laser sintering (SLS) uses a high power laser or another focused heat source to sinter or weld small particles of plastic, metal, or ceramic powders into a mass representing the 3-dimensional object to be formed.

Fused deposition modelling (FDM) and related techniques make use of a temporary transition from a solid material to a liquid state, usually due to heating. The material is driven through an extrusion nozzle in a controlled way and deposited in the required place as described among others in U.S. Pat. No. 5,141,680.

Foil-based techniques fix coats to one another by means of gluing or photo polymerization or other techniques and cut the object from these coats or polymerize the object. Such a technique is described in U.S. Pat. No. 5,192,539.

Typically AM techniques start from a digital representation of the 3-D object to be formed, e.g. a guide according to any of the embodiments of the present invention. Generally, the digital is sliced into a series of cross-sectional layers which can be overlaid to form the object as a whole. The AM apparatus uses this data for building the object on a layer-by-layer basis. The cross-sectional data representing the layer data of the 3-D object may be generated using a computer system and computer aided design and manufacturing (CAD/CAM) software. A common feature of such techniques is that objects are typically built layer by layer.

A selective laser sintering (SLS) apparatus is particularly preferred for the manufacture of the surface that is the negative form of a body part (as well as its support) from a computer model. It should be understood however, that various types of additive manufacturing and tooling may be used for accurately fabricating these surfaces and supports including, but not limited to, stereolithography (SLA), Fused Deposition Modeling (FDM) or milling.

The support for such a surface may be manufactured in different materials. Preferably, only materials that are biocompatible with the human body are taken into account. In the case SLS is used as a AM technique, the support for the surface may be fabricated from a polyamide such as PA 2200 as supplied by EOS, Munich, Germany or Duraform Pa. from 3D Systems, South Caroline, USA, or any other material known by those skilled in the art may also be used. While the invention has been shown and described with reference to preferred embodiments, it will be understood by those skilled in the art that various changes or modifications in form and detail may be made without departing from the scope and spirit of this invention.

1.-17. (canceled)

18. A surgical, therapeutic or diagnostic guide (1) comprising:
a rigid structure, and
at least one functional guidance element (13),
first means (6) for positioning and holding the rigid structure by compression, the first means for positioning and holding the rigid structure by compression being at a first location on the rigid structure and including a first surface (14), that is, before application, manufactured according to a specific patient, having a negative form of and registering with a first part of the body of the specific patient, and further including a first compression means for holding the first surface against the first part of the body of the specific patient, and
second means (2) for positioning and holding the rigid structure by compression about a second part of the body of the specific patient, the second means for positioning and holding the rigid structure by compression being at a second location on the rigid structure remote from the first location, the second means for positioning and holding including a second surface (12), that is, before application, manufactured according to the specific patient, having a negative form of and registering with at least a portion of the second part of the body of the specific patient, and further including a second compression means for holding the second surface against the second part of the body of the specific patient;
wherein the first and/or second means for positioning and holding the rigid structure by compression is adapted for positional clamping the first or second surface respectively to the first or second part of the body, respectively.

19. The guide according to claim 18 wherein the first and/or second surfaces are formed by additive or layered manufacturing.

20. The guide according to claim 19 wherein said additive or layered manufacturing is based on at least one medical image of the specific patient.

21. The guide according to claim 18, wherein the rigid structure and the first and/or second means for positioning and holding the rigid structure by compression are non-invasive.

22. The guide according to claim 18, wherein the first and/or second means for positioning and holding the rigid structure are adapted to apply compression by a circumferential clamp or a scissor clamp.

23. The guide according to claim 18, wherein the at least one functional guidance element is adapted for execution of a therapeutic, diagnostic or surgical act.

24. The guide according to claim 23, wherein the therapeutic, diagnostic or surgical act is selected from insertion of a biopsy needle, placement of reference pins, drilling of holes, surgical cutting, osteotomy cutting or wherein the at least one functional guidance element comprises an interface with an electronic surgical navigation system or is a reference for an electronic surgical navigation system.

25. The guide according to claim 18, wherein the rigid structure and the first and second means (6, 2) are made of plaster, a stiff moulding or modeling material, or expandable foam.

26. The guide according to claim 18, wherein negative tolerances or cushioning components are provided on the rigid structure.

27. The guide according to claim 18, further comprising a number of radio-opaque construction elements.

28. The guide according to claim 18, further comprising inflatable areas.

29. The guide according to claim 18, wherein the at least one functional guidance element is attached directly or indirectly to the rigid structure.

30. A method of attaching a surgical, therapeutic or diagnostic guide (1) to a body of a patient, the guide (1) having a rigid structure and being adapted for use with at least one functional guidance element (13), the method comprising the steps of:
positioning and compression holding the rigid structure at a first location on the rigid structure at a first part of the body of the patient by a first surface (14), that is patient specific, having a negative form of a first part of the body of the patient,
manufacturing the first surface (14) according to the first part of the body the patient before application,
positioning and holding the rigid structure by compression at a second location on the rigid structure remote from the first location about a second part of the body of the patient by a second surface (12), that is patient specific, having a negative form of at least a portion of the second part of the body of the patient,
manufacturing the second surface (12) according to at least a portion of the second part of the body of the patient before application, and
wherein the positioning and compression holding steps are non-invasive.

31. The method according to claim 30, further comprising scanning the patient with the surgical, therapeutic or diagnostic guide in place and adding the at least one functional guidance element after the scanning step.

32. The method according to claim 30 further comprising determining said first location and/or said second location and/or a location of said at least one guidance element on said rigid structure using surgical planning techniques that use at least one medical image of the patient.

33. The method of claim 31, wherein the scanning step is selected from the group of an MRI, a CT-Scan, an Ultrasound scan, and a PET scanning step.

34. A method of making the guides according to claim 18, the method including an additive manufacturing technique such as rapid prototyping or layered manufacture.
